(19) United States
(12) Patent Application Publication
(10) Pub. No.: US 2026/0207258 A1
(43) Pub. Date: Jul. 23, 2026

(54) DEVICE FOR ASSISTING IN THE PLANNING OF A MINIMALLY INVASIVE PROCEDURE

(71) Applicant: QUANTUM SURGICAL, Montpellier (FR)

(72) Inventors: Estanislao OUBEL, Montpellier (FR); Yann LE MEUR, Saint-Brès (FR); Lucien BLONDEL, Montpellier (FR); Bertin NAHUM, Castelnau-le-Lez (FR); Fernand BADANO, LYON (FR)

(21) Appl. No.: 19/139,806

(22) PCT Filed: Dec. 14, 2023
(86) PCT No.: PCT/EP2023/085766

§ 371 Date: Jun. 16, 2025

(30) Foreign Application Priority Data
Dec. 16, 2022 (FR) FR2213696

(51) Int. Cl. A61B 34/10; A61B 34/00; A61B 34/30

(52) U.S. Cl. CPC A61B 34/10; A61B 34/25; A61B 34/30; A61B 2034/105; A61B 2034/107; A61B 2034/252

(57) ABSTRACT

The invention relates to a data processing device implementing a method for helping in the selection of at least one optimal trajectory for a minimally invasive medical procedure. The device is in particular configured to identify, in a three-dimensional anatomical model, a target point to be reached in a region to be treated within an anatomy of interest of a patient, and to perform at least one iteration comprising the steps of: —determining a sampling region and a sampling resolution of candidate entry points, —eliminating candidate trajectories according to at least one predetermined validity criterion, —for each remaining candidate trajectory, calculating at least one cost function from a plurality of quantified characteristics, —displaying candidate trajectories on the anatomical model with a means for rapidly visualising the cost function, —selecting an optimal trajectory from the displayed candidate trajectories.

### FIELD OF THE INVENTION

The present patent application relates to the field of planning a minimally invasive medical procedure, possibly one assisted by a medical robot. In particular, a data-processing device for implementing a method for assisting with selection of an optimal path for a minimally invasive medical procedure within an anatomy of interest of a patient is provided.

### PRIOR ART

To prepare for a minimally invasive medical procedure aimed at reaching a target anatomical region in an anatomy of interest of a patient with a medical instrument, a practitioner generally plans the procedure using a pre-operative or intra-operative medical image. The minimally invasive medical procedure may in particular be intended to achieve biopsy or ablation of a tumor in an organ or bone, to perform a vertebroplasty or a cementoplasty, or even to stimulate a particular anatomical region. The anatomy of interest may for example be a lung, a kidney, the liver, the brain, a vertebra, a tibia, a knee, etc. The medical instrument may be a needle, an electrode, a probe, etc.

The pre-operative or intra-operative medical image is for example obtained via computed tomography, magnetic resonance imaging, ultrasound, or positron emission tomography.

When planning the procedure, the practitioner defines a target point in a region to be treated of the anatomy of interest. The practitioner also defines a point of entry of the medical instrument on the skin of the patient. These two points then define a path that the medical instrument must follow in order to carry out the medical procedure.

Depending on the type of procedure, certain constraints must be met. For example, it may be important for the medical instrument not to pass through a high-risk anatomical structure (organ, bone or blood vessel for example).

The path is generally defined empirically based on the knowledge of the practitioner and on information she or he is able to gather from the pre-operative image. However, this path is not necessarily optimal, i.e. the one that ensures effective treatment of the patient with minimal risk.

For a given procedure, there are theoretically an infinite number of possible paths. However, the practitioner must select a path in a limited and sometimes very short amount of time, in particular when the procedure is planned just before it is carried out.

In addition, this task must be performed based on volume images of the patient, and therefore in three dimensions, whereas conventional methods for presenting such images deliver two-dimensional sections of the volume, which makes it even more difficult for the practitioner to find the optimal path. Furthermore, when the procedure requires a plurality of paths to be defined (for example with a view to insertion of a plurality of needles), it becomes even more complex for the practitioner to plan it mentally.

Ultimately, the quality of path optimization is highly dependent on the practitioner and on the time that she or he is able to allocate to this task.

Methods for evaluating a path proposed by a practitioner, for example in light of the risk of crossing a high-risk region with the medical instrument, already exist. Such solutions do not allow the practitioner to rapidly find an optimal or near-optimal path for the medical procedure in question.

Planning methods in which the point of entry, target point and/or path are suggested to the practitioner automatically also already exist. However, these solutions generally do not give the user flexibility to view the optimality of a large number of paths, and to choose a different path that may be sub-optimal, but that is preferable for reasons that may vary from one patient to another and that are not necessarily taken into account by the optimality criteria in question.

### DESCRIPTION OF THE INVENTION

The objective of the solutions provided in the present patent application is to remedy all or some of the drawbacks of the prior art, in particular those described above.

To this end, and according to a first aspect, a data-processing device comprising a processor, a computer memory and a display screen is provided. The computer memory comprises program-code instructions that, when they are executed by the processor, configure the processor to implement a method for assisting with selection of at least one optimal path for a minimally invasive medical procedure within an anatomy of interest of a patient. The processor is configured to:
obtain a three-dimensional anatomical model of the patient from previously acquired medical images of the patient, the anatomical model comprising a representation of the body envelope and of the anatomy of interest of the patient,
identify in the anatomical model at least one target point to be reached in a region to be treated within the anatomy of interest of the patient,
perform at least one iteration in which the processor is configured to:
   determine a sampling region and a sampling resolution for sampling candidate points of entry, in the anatomical model, on the body envelope of the patient, each candidate point of entry belonging to the sampling region, the sampling resolution being representative of a minimum distance separating two candidate points of entry, each pair formed by the target point and a candidate point of entry forming one candidate path,
   remove candidate paths in light of at least one predetermined validity criterion,
   for each remaining candidate path, quantify each of a plurality of predetermined characteristics, and compute at least one cost function from the quantified characteristics,
   display, on the display screen, superposed on the anatomical model, at least some of the remaining candidate paths with, for each displayed candidate path, a means for rapidly viewing a value of the cost function,
   obtain the selection, by a user, for said at least one target point, of a path considered to be optimal among the displayed candidate paths.

Such provisions make it possible to suggest to a practitioner a set of optimal or near-optimal paths for a minimally invasive procedure, based on a three-dimensional anatomical model of the patient. The suggested paths take into account constraints of various types (for example practical constraints, performance-related constraints and/or safety constraints). The practitioner is able to choose a path that may be sub-optimal in respect of a predefined criterion, but that is preferable in respect of constraints not addressed by that criterion. The invention further allows for rapid and optimized exploration of all the solutions, with a view to determining candidate paths. It allows the user to rapidly view various candidate paths and their optimality criteria. The proposed solution is flexible as it makes it possible to adapt to the specific needs of the procedure. The practitioner is further able to update the optimality criteria (cost function) in light of user feedback (evaluation of a path selected for the procedure).

In particular embodiments, the device may further comprise one or more of the following features, implemented alone or in any technically possible combination.

In particular embodiments, the processor is configured to perform at least two iterations. For an iteration of rank n, n being an integer strictly greater than one:
the sampling region of the iteration of rank n is defined to be smaller than the sampling region defined for the iteration of rank (n−1), and to include the candidate point of entry corresponding to the path selected in the iteration of rank (n−1), and
the sampling resolution of the iteration of rank n is defined to be finer than the sampling resolution defined for the iteration of rank (n−1).

In particular embodiments, the sampling resolution of the iteration of rank n is defined in light of a variability of the cost function computed for the iteration of rank (n−1) in the vicinity of the path selected in the iteration of rank (n−1).

In particular embodiments, the sampling region of the iteration of rank n is defined in light of a variability of the cost function computed for the iteration of rank (n−1) in the vicinity of the path selected in the iteration of rank (n−1).

In particular embodiments, to sample the candidate points of entry, the processor is configured to determine, in the anatomical model, on the body envelope of the patient, a set of points defined by a spherical coordinate system centered on the target point. Each point is defined by a distance r_{i,j} from the target point and by two angles αᵢ and βᵢ the angles αᵢ and βⱼ being defined such that, for indices i and j corresponding to strictly positive integers:

$${α}_{i , j} = {α}_{i - 1 , j} + \frac{R}{{r}_{i , j}} \qquad [ Math . 1 ]$$

$$and$$

$${β}_{i , j} = {β}_{i , j - 1} + \frac{R}{{r}_{i , j}} \qquad [ Math . 2 ]$$

where R is representative of a minimum distance separating two candidate points of entry on the body envelope of the patient.

In particular embodiments, one validity criterion allows, for a given candidate path, at least one of the following to be verified:
a validity of the length of the candidate path with respect to a medical instrument envisioned for the procedure,
an ability to configure a robotic arm equipped with a medical instrument so that said medical instrument will be able to follow the candidate path,
the presence of objects with which the patient is equipped obstructing the candidate path,
an intersection of the candidate path with at least one critical anatomical structure internal to the body of the patient.

In particular embodiments, a plurality of validity criteria are considered to remove candidate paths, and the various validity criteria are evaluated in an order defined in light, for each validity criterion, of an estimated computation time required to assess said validity criterion.

In particular embodiments, the plurality of characteristics of a candidate path comprises at least one of the following characteristics:
a minimum distance between the candidate path and a critical anatomical structure internal to the body of the patient,
a length of the intersection between the candidate path and the anatomy of interest,
a minimum angle of incidence between the candidate path and an anatomical interface crossed by the candidate path,
an estimated value representative of the stability, under the effect of its own weight, of a medical instrument positioned up to the target point along the candidate path,
a minimum distance between an envelope of the region to be treated and an envelope of an ablation region estimated for the candidate path,
an angular difference between the candidate path and a major axis of the region to be treated.

In particular embodiments, a plurality of cost functions are computed from the quantified characteristics, each cost function being computed with a different set of respective weightings assigned to the various quantified characteristics, and the processor is configured to obtain an indication, by a user, of the cost function to be considered when displaying candidate paths.

In particular embodiments, the means for rapidly viewing the value of the cost function for each displayed candidate path comprises a color code associating various respective colors with various values of the cost function.

In particular embodiments, the processor is configured to display the quantifications determined for the characteristics of the path selected by the user.

In particular embodiments, the processor is configured to obtain, for at least one displayed candidate path, an evaluation by the user of said candidate path, and to update the cost function in light of said evaluation.

In particular embodiments, the processor is configured to:
determine in the anatomical model, after the procedure, an actual path followed by a medical instrument during the procedure, from medical images of the patient acquired at a time when the medical instrument is in place,
determine a value of the cost function for the actual path,
compute a difference between the value of the cost function of the candidate path selected for the procedure and the value of the cost function of the actual path.

In particular embodiments, the processor is configured to compare the computed difference with a predetermined threshold and to display an indication relating to the value of the computed difference.

In particular embodiments, the processor is configured to:
obtain an evaluation of the actual path by the user,
determine, in light of the computed difference and of the obtained evaluation, a statistical value representative of the variability of the cost function,
remove candidate paths that are considered to be equivalent given the statistical value thus obtained.

### OVERVIEW OF THE FIGURES

The invention will be better understood on reading the following description, which is given by way of completely non-limiting example, and with reference to FIGS. 1 to 7 which show:

FIG. 1 a schematic representation of a data-processing device according to the invention,

FIG. 2 a schematic representation of the main steps of a method for assisting with selection of an optimal path for a medical procedure,

FIG. 3 a schematic representation of the definition of an orbital angle and of a cranio-caudal angle with a view to defining a sampling region,

FIG. 4 a schematic representation of a set of candidate points of entry, in the anatomical model, on the body envelope of the patient,

FIG. 5 a schematic representation of the display of a set of candidate paths for the medical procedure, with their optimality criterion displayed via a scale, here a grayscale,

FIG. 6 a schematic representation of additional steps for taking into account an evaluation by a user of a candidate path,

FIG. 7 a schematic representation of additional steps for taking into account a difference between the selected path and the path actually followed by the medical instrument,

FIG. 8 a schematic representation of a bone to be treated and of two candidate paths to reach a target point in the bone.

In these figures, identical references from one figure to another denote identical or analogous elements. For reasons of clarity, the elements represented are not necessarily on the same scale, unless otherwise stated.

### DETAILED DESCRIPTION OF AT LEAST ONE EMBODIMENT OF THE INVENTION

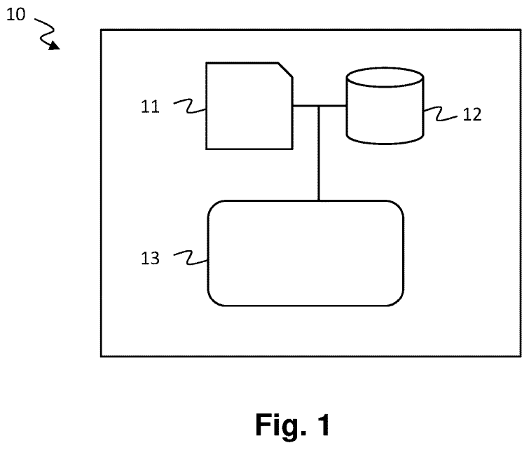

FIG. 1 shows a data-processing device 10 according to the invention. The data-processing device 10 comprises at least one processor 11, at least one computer memory 12 and at least one display screen 13.

The computer memory 12 comprises program-code instructions that, when they are executed by the processor 11, configure the processor 11 to implement a method for assisting with selection of at least one optimal path that a medical instrument must follow to perform a minimally invasive medical procedure within an anatomy of interest of a patient.

The minimally invasive medical procedure may in particular be intended to achieve biopsy or ablation of a tumor in an organ or bone, to treat a bone disease, to perform a vertebroplasty or a cementoplasty, or even to stimulate a particular anatomical region. The anatomy of interest may correspond to an organ or to a bone, for example a lung, a kidney, the liver, the brain, a vertebra, a tibia, a femur, a hip, a knee, the pelvic bones, the pelvis, etc. The medical instrument may be a needle, an electrode, a probe, a drill, a trocar, a screw, etc.

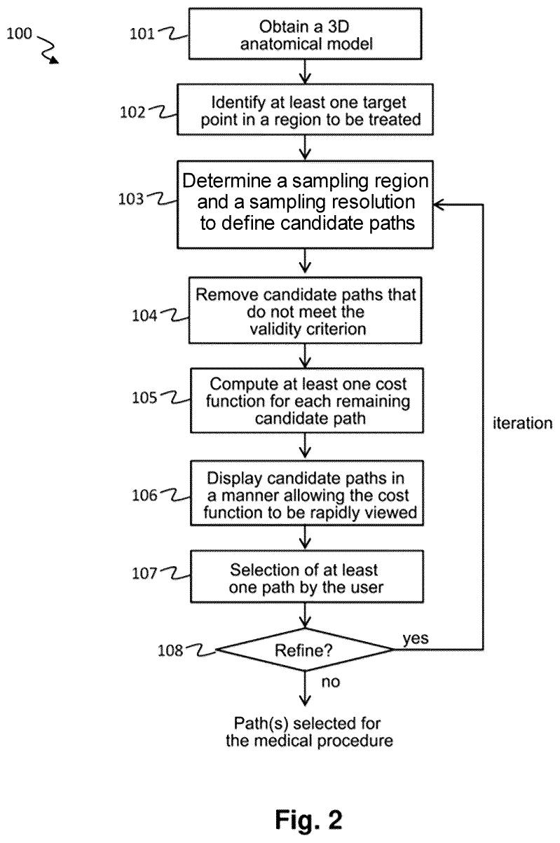

FIG. 2 shows the main steps of a method 100 implemented by the processing device 10 to assist with selection of an optimal path for a minimally invasive medical procedure.

As illustrated in FIG. 2, the method 100 comprises a step 101 of obtaining a three-dimensional anatomical model of the patient. The anatomical model in particular comprises a representation of the body envelope (or part of the body envelope) and of the anatomy of interest of the patient.

The anatomical model is obtained from previously acquired medical images of the patient. The medical images used to generate the anatomical model are for example obtained via computed tomography, magnetic resonance imaging, ultrasound, or positron emission tomography (any medical imaging technique allowing a volume to be reconstructed in three dimensions may be used). A plurality of images are generally required to generate a three-dimensional anatomical model. Alternatively, it is possible to generate a three-dimensional anatomical model from a single image and a statistical model of the patient.

The anatomical model may be generated directly by the processing device 10, but it may also optionally be generated by another separate device and transmitted to the processing device 10 via communication means.

Conventionally, three-dimensional modeling is for example carried out using methods for segmenting, in medical images, anatomical structures considered relevant by clinical practice. The results of the segmentations may take the form of binary images or of surfaces. For example, in the context of minimally invasive ablation of a liver tumor, it may be relevant to segment the liver, the tumor, the lungs, the gallbladder, the bile ducts, the organs of the digestive system, the bones, certain blood vessels (those of significant diameter), and the body envelope of the patient. According to another example, in the context of a minimally invasive procedure on a bone, it may also be relevant to segment the external envelope of the bone to be treated, its cortical part (cortical bone corresponds to the particularly rigid peripheral part of the bone; it is the “external shell” of the bone, i.e. a relatively thick wall of the bone) and/or its cancellous part (cancellous bone corresponds to the bone tissue that forms the porous part of the bone, which is located under the cortical bone). This three-dimensional anatomical modeling may be represented mathematically in the following way:

$$M = {{LJ}^{N}}_{i = 1} {S}_{i} \qquad [ Math . 3 ]$$

where M is the three-dimensional anatomical model and S, are the representations of the segmentations taken into account to generate the model, the various segmentations all being represented in the same anatomical frame.

Segmenting methods are generally automatic artificial-intelligence methods (no user input is required), semi-automatic artificial-intelligence methods (a single user input such as a point or segment is then required), or interactive artificial-intelligence methods (segmentation results are iteratively corrected by a user). Manual segmentation could also be envisioned, but the time required to perform such segmentation means this alternative is of little relevance in a clinical context.

Among artificial-intelligence methods, convolutional neural networks are currently the most effective. Other methods may however be advantageous alternatives, such as for example methods based on random forests, on k-means clustering, on watershed segmentation, on an active contour model, on the level-set method, etc.

As illustrated in FIG. 2, the method 100 comprises a step 102 of identifying, in the anatomical model, a target point to be reached in the region to be treated (a tumor for example) within the anatomy of interest of the patient (the liver for example). It should be noted that it is also possible to envision the case where a plurality of target points must be considered (for example when a plurality of paths must be selected for insertion of a plurality of needles). In this case, this set of target points may be determined from a “virtual” target point and from a geometrical arrangement that the target points must have with respect to one another and with respect to the virtual target point (for example the target points must correspond to the vertices of a particular geometric shape centered on the virtual target point, or indeed have a particular spatial distribution around the virtual target point). The virtual target point and the geometrical arrangement that the target points must have are input data provided by the user.

The region to be ablated may in particular be segmented automatically or semi-automatically. It is possible to use manual segmentation or correction tools depending on the desired quality. Once a satisfactory segmentation of the region to be treated has been obtained, the position of a target point to be reached may be estimated. It will be noted that the region to be treated may be segmented by the processing device 10, or indeed it may have been previously segmented in the anatomical model by another separate device then transmitted to the processing device 10 via communication means.

The segmentation of the region to be treated may for example be used to determine a target point automatically (for example through computation of a barycenter or center of an encapsulating sphere or ellipsoid determined using parameters that are known already). Alternatively, when the procedure involves insertion of a plurality of medical instruments, it is conceivable to use the segmentation to determine a set of a plurality of target points, for example in light of a virtual target point (for example corresponding to the center of the region to be treated), of a geometrical arrangement that the target points must have, and/or of a desired ablation coverage (specifically, it is possible, for a given medical instrument, to predict an ablation zone liable to be obtained around the target point, in order to ensure that it covers a region that it is desired to ablate).

Depending on the type of treatment envisioned (radio-frequency ablation, microwave ablation, cryotherapy or electroporation, biopsy, cementoplasty, etc.), the target point may be located within or on the periphery of the region to be treated. The position of the target point may optionally be estimated in this way in light of a model of the ablation zone liable to be obtained when the medical instrument is positioned at the target point.

The candidate paths of the procedure will be determined from the one or more target points thus defined and from a set of candidate points of entry on the skin of the patient. The set of points of entry on the skin therefore forms an initial domain based on which an optimal path must be identified. Specifically, each pair formed by a target point and a candidate point of entry forms one candidate path. It is therefore necessary to identify candidate points of entry on the skin of the patient. This may be achieved via methods such as Otsu's method, which is highly suitable for classifying voxels in the case of bimodal brightness distributions. However, in continuous space there are an infinite number of candidate points of entry. Even in the discrete domain, for the image resolutions used in clinical practice, there are a very high number of candidate points of entry. It is therefore preferable to carry out sampling in order to reduce the number of candidate points.

For this purpose, and as illustrated in FIG. 2, the method 100 comprises a step 103 of determining a sampling region and a sampling resolution for sampling candidate points of entry, in the anatomical model, on the body envelope of the patient.

The sampling resolution is representative of a minimum distance separating two candidate points of entry. A compromise must be found between the fineness of the sampling and the computation time required to evaluate the respective candidate paths associated with each candidate point of entry. A “brute force” approach with very fine sampling of the candidate points of entry would require path evaluation to be computed very rapidly, which would lead to strong constraints (limitation of optimization criteria and/or use of expensive computing means). In addition, the user would need to view a large amount of information.

It may therefore be advantageous to consider a sampling resolution such that two contiguous paths are sufficiently different from each other. The sampling resolution may be determined by default (for example a minimum distance between two points of entry of at least five millimeters on the body surface of the patient may be required), but it may also be adjusted by the user as required (compromise between computation time and the desired precision).

To sample the candidate points of entry, it is in particular conceivable to determine, in the anatomical model, on the body envelope of the patient, a set of points defined by a spherical coordinate system centered on the target point (when a plurality of target points are considered, it is conceivable to process each target point in succession with the aim of determining an optimal path individually for each of the target points; alternatively, it is possible to process a set of target points together, and in this case it is possible to sample the candidate points of entry with respect to a “virtual” target point corresponding for example to a point equidistant from each of the target points). Each point may then be defined by a distance r_{i,j} from the target point and by two angles αᵢ and βⱼ. The sampling region may then correspond to the intersection of the body envelope with a cone of angular aperture (described by angles αₘₐₓ and βₘₐₓ) centered on the target point. The computation is for example initialized with a first point on the skin corresponding to a path with α₀=β₀=0; the distance between this point and the origin of the coordinate system (the target point) is denoted r_{0,0}. The sampling may be obtained by computing angles αᵢ and βᵢ (in radians) such that an increment between each angle corresponds to an arc length on the body envelope of the patient corresponding to a predefined resolution parameter. The angles αᵢ and βⱼ may in particular be defined in such a way that, for indices i and j corresponding to strictly positive integers:

$${α}_{i , j} = {α}_{i - 1 , j} + \frac{R}{{r}_{i , j}} \qquad [ Math . 1 ]$$

$$and$$

$${β}_{i , j} = {β}_{i , j - 1} + \frac{R}{{r}_{i , j}} \qquad [ Math . 2 ]$$

where R is a predefined resolution parameter representative of a minimum distance separating two candidate points of entry on the body envelope of the patient. The angles α and β are incremented until they cover the cone of desired aperture.

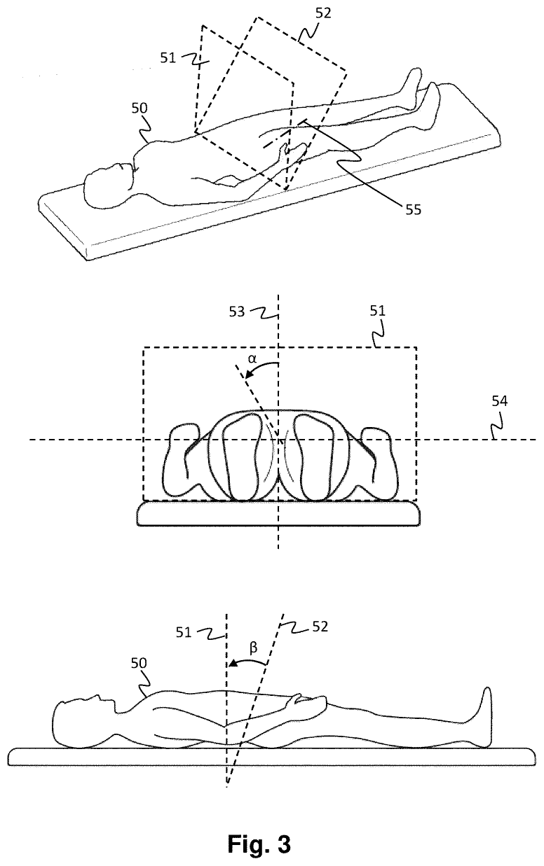

As illustrated in FIG. 3, the angle α may correspond to an orbital angle measured with respect to an antero-posterior axis 53 in a transverse plane 51 of the patient 50. The transverse plane 51 is defined by the target point, the antero-posterior axis 53 and a transverse axis 54 of the patient. The angle β may correspond to a cranio-caudal angle made by the transverse plane 51 and an oblique plane 52. The oblique plane 52 is defined by the target point, the transverse axis 54, and the candidate path 55 formed by the target point and the point of entry in question.

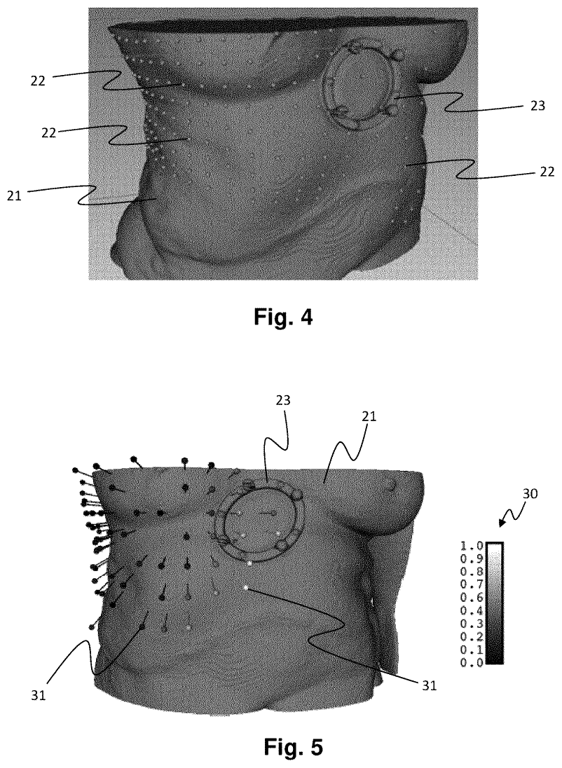

FIG. 4 schematically shows a set of candidate points of entry 22 in the anatomical model on the body envelope 21 of the patient (the candidate points of entry 22 form a “mesh” on the body envelope 21 of the patient).

As indicated above, it is advantageous to reduce the number of candidate paths to limit the overall computation time of the method for selecting an optimal path. For this purpose, and as illustrated in FIG. 2, the method 100 comprises a step 104 of removing certain candidate paths in light of one or more predetermined validity criteria.

It is advantageous to consider a plurality of different validity criteria and to evaluate them in a predefined order. This order is defined in light of an estimated computation time required to evaluate each validity criterion.

It is a question of applying successive simple filters in order to reduce the number of candidate paths. These filters are simple to compute and allow certain impossible paths to be quickly rejected. These filters are applied from the simplest to the most complex, in terms of computation time, so that the last filters are applied to the lowest possible number of candidate paths.

By way of non-limiting example, the following validity criteria may be successively taken into account.

In an initial stage, if the medical procedure is assisted by a robot comprising a robotic arm equipped with a medical instrument, it is possible to remove candidate paths for which the robotic arm cannot be configured to make the medical instrument follow the path in question. Once the sampling has been carried out, the candidate paths are filtered based on prior knowledge of the ability of the robot to access the orbital and cranio-caudal angles and the target point in question. This stage depends on the model of robot used, on the geometry of the tools used to guide the medical instrument, and on the position of the target point. These parameters are known when the candidate paths are determined, and it is therefore possible to greatly reduce the number of candidate paths.

In a second stage, it is possible to remove candidate paths the path length of which is greater than the length of the medical instrument envisioned for the procedure.

In a third stage, it is possible to remove candidate paths that are obstructed by an object with which the patient is equipped. As illustrated in FIG. 4, such an object may for example be a patient reference 23 used by an optical navigation system to guide the robotic arm. According to another example, it could also be a catheter. Through segmentation of the patient and of the objects with which they are equipped for the procedure, and prior knowledge of the three-dimensional geometry of the robotic arm, it is possible to determine paths leading to collisions between the robotic arm and objects with which the patient is equipped for the procedure. Such paths may be removed.

In a fourth stage, it is possible to remove candidate paths that intersect with at least one critical anatomical structure internal to the body of the patient. By critical anatomical structure, what is for example meant is an organ different from the anatomy of interest (for example the lungs, spleen, gallbladder or kidneys if the anatomy of interest is the liver) or high-risk vascular structures (arteries, veins, bile ducts, digestive tract). In general, segmentations are not represented parametrically, thus, to detect a collision between a high-risk anatomical structure and the path, it is possible to finely sample the latter and determine the segmentation value for each point of the sample. A distance between points on the path equal to half the resolution of the image in the acquisition direction, which is typically of the order of one millimeter, is sufficient for this purpose.

The validity criteria listed above by way of example may apply both to a minimally invasive procedure on a soft organ and a minimally invasive procedure on a bone.

As illustrated in FIG. 2, the method 100 comprises a step 105 of computing at least one cost function, for each remaining candidate path (i.e. for each candidate path that was not removed in the removing step 104) based on a plurality of characteristics relating, for example, to the safety or performance of the procedure. These characteristics are quantified, meaning that a measured and standardized value is assigned to each characteristic in question (in other words, each quantified characteristic corresponds to a metric representative of a safety or performance criterion).

By way of non-limiting example, all or some of the following characteristics may be taken into account when computing a cost function. Unless otherwise stated, each of these characteristics may be taken into account for a minimally invasive procedure on a soft organ or for a minimally invasive procedure on a bone.

According to a first example, it is possible to take into account a minimum distance between the candidate path and a critical anatomical structure internal to the body of the patient. For this purpose, the anatomical model may comprise a segmentation of the internal critical anatomical structures. A distance between each point of the path and a given anatomical structure may then be computed using a distance transform (also called a distance map) of the associated segmentation. When this transformation is applied to a binary image, the result is a new image where the value of each voxel corresponds to the shortest distance between the voxel and the edge of the image. The minimum distance between the candidate path and an internal critical anatomical structure corresponds to a safety margin that must account for factors affecting the imprecision of the medical robot and/or biomechanical variables that cannot be controlled during insertion of the medical instrument.

According to another example, it is conceivable to take into account a length of the intersection between the candidate path and the anatomy of interest. This characteristic is related to a safety criterion of the medical procedure. Specifically, it may be necessary for the medical instrument to pass a minimum distance through the anatomy of interest to prevent tumor cells from being spread outside of the anatomy of interest when it is removed. This characteristic is also related to a performance criterion because the longer the intersection between the candidate path and the anatomy of interest, the better the stability of the medical instrument in the anatomy of interest during the procedure. There is therefore a compromise to be found in light of the targeted clinical objectives; this compromise may be specific to each procedure. In the case where the objective of the minimally invasive procedure is to insert a screw into a bone, the minimum distance of penetration by the screw into the bone is also relevant to the stability of the screw in the bone.

According to another example, it is possible to take into account a minimum angle of incidence between the candidate path and an anatomical interface crossed by the candidate path. When inserting a medical instrument, a needle for example, during a minimally invasive procedure, the instrument may pass through a certain number of interfaces, such as the skin or walls of certain organs (such as the liver capsule). The angle of incidence of the instrument with respect to these interfaces may have an impact on instrument placement effectiveness (potential deflection of the instrument or movement of the organ). This parameter also has an impact on safety since an angle of incidence that is too tangent to an anatomical interface may promote the formation of a hematoma. Computation of the angle of incidence requires the normal to the surface at the point of entry of the path to be known. Approaches based on surface meshing exist but they can be expensive, which compromises their use in a clinical context. In order to reduce computation time, the normal may be computed as the gradient of the distance transform of the segmentation evaluated at the point of entry. It is advisable to compute the gradient via convolution with the derivative of a Gaussian in order to obtain estimates of the normal that are more robust to noise (sensitivity to noise is one disadvantage of finite-difference methods). In the case of a procedure on a bone, too small an angle of incidence may increase the risk of the medical instrument slipping on the bone.

According to another example, it is possible to take into account an estimated value representative of the stability, under the effect of its own weight, of a medical instrument positioned up to the target point along the candidate path. This value may in particular be computed in light of the insertion length of the path in question: the shorter the insertion length, the more easily the medical instrument will be able to move under the effect of its own weight or external forces. This value may also be computed using a biomechanical model providing information on the viscoelasticity of the organs passed through, for example. Paths having the least risk of causing the medical instrument to move under its own weight will be preferred. This characteristic is more relevant for a soft organ than for a bone.

According to another example, when the minimally invasive procedure corresponds to ablation, it is possible to take into account a minimum distance between the envelope of the region to be treated and the envelope of an ablation region estimated for the candidate path. This minimum distance corresponds to an estimated ablation margin. For a given medical instrument, models of ablation zones are available. These models allow the theoretical region of coverage obtained for each path to be estimated. For example, it is advantageous to optimize coverage of a cancerous region by the ablation (maximize the margin between the ablated region and the cancerous region) while minimizing damage to the healthy parenchyma and nearby organs. Generally, the models of ablation zones are ellipsoidal and off-centered with respect to the distal end of the instrument. The shape and orientation of the tumor are important aspects to consider (the tumor is not necessarily spherical). Therefore, in order to obtain a better coverage of the tumor, certain paths may be preferred depending on their orientation and on the position of the distal end of the instrument at the end of the path.

According to another example, it is also possible to take into account an angular difference between the candidate path and a major axis of the region to be treated. Specifically, in practice, to ablate a tumor, it is advisable to attack the tumor along its major axis because the treatment is more effective on the axis of the medical instrument.

According to another example, when the minimally invasive procedure corresponds to ablation, it is also possible to take into account a coverage value of the region to be treated. This coverage value may be estimated in light of a segmentation of the region to be treated and of an estimate of an ablation zone liable to be obtained with the candidate path in question. Specifically, minimum ablation margins may be required to minimize the recurrence rate, for example in the case of ablation of a tumor. In the case of a bone tumor, another important factor is that the ablation zone remains inside the bone (coverage constraints are then dictated by the anatomy and position of the lesion).

According to another example, when the anatomy of interest is a bone, it is also possible to take into account a minimum distance between a part of the candidate path and the cortical layer of the bone, for the part of the path that is inside the bone. Specifically, it is advantageous to maximize the minimum distance between the part of the candidate path that is inside the bone and the cortical layer of the bone, in order to minimize the risk of the medical instrument, after insertion thereof into the bone, touching the cortical layer of the bone again. Specifically, the actual path followed by the medical instrument may differ from a selected theoretical path as a result of imprecision in the placement of the medical instrument or of biomechanical effects (deflection of the medical instrument, movement of the anatomy of interest due to the patient breathing or due to effects associated with insertion of the instrument, etc.). Maximizing the minimum distance between the part of the candidate path that is inside the bone and the cortical layer of the bone amounts to maximizing a margin of safety of the candidate path.

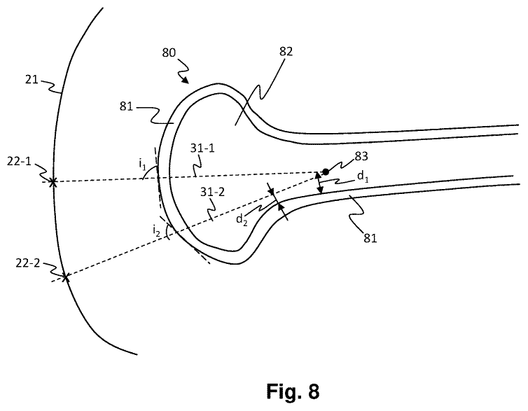

FIG. 8 schematically shows a bone 80 to be treated with the cortical layer 81 of the bone and the cancellous part 82 of the bone. Reference 83 designates a target point that a medical instrument must reach to treat the bone 80. FIG. 8 also shows two candidate paths 31-1 and 31-2 for reaching the target point 83 from two candidate points of entry 22-1 and 22-2 on the body surface 21 of the patient, respectively. In the example shown in FIG. 8, the candidate path 31-1 could be preferred to the candidate path 31-2 because the minimum distance d₁ separating the part of candidate path 31-1 that is inside the bone 80 from the cortical layer 81 of the bone is larger than the minimum distance d₂ separating the part of candidate path 31-2 that is inside the bone 80 from the cortical layer 81 of the bone.

According to another example, when the anatomy of interest is a bone, it is also possible to take into account an angle of incidence between the candidate path and the wall of the bone. Specifically, it is advantageous for the candidate path to have an angle of incidence as close as possible to a right angle (angle of 90°) to prevent the medical instrument from sliding over the wall of the bone during its insertion (this could damage the bone during insertion of the medical instrument and/or cause the medical instrument to deviate from the selected path). What is meant by “angle of incidence” is the angle made by the candidate path and the bone at the interface where the path penetrates into the interior of the bone.

In the example shown in FIG. 8, candidate path 31-1 could be preferred to candidate path 31-2 because the angle of incidence i₁ made by candidate path 31-1 and the wall of the bone 80 is closer to a right angle than the angle of incidence i₂ made by candidate path 31-1 and the wall of the bone 80.

According to another example, when the anatomy of interest is a bone, it is also possible to take into account an estimate of a risk of the bone fracturing as a result of a procedure using the candidate path in question. For example, when the procedure aims to place a screw into a bone to consolidate it, it is possible to use a biomechanical model capable of modeling the structure of the bone (with solid regions and more fragile regions of the bone) and mechanical stresses undergone by the bone (for example depending on patient measurements) in order to estimate a risk of the bone fracturing as a result of the screw being inserted along the candidate path in question. This risk may optionally be compared with a risk of the bone fracturing should the procedure not be carried out (i.e. without the consolidation screw), in order to estimate how much the procedure decreases the risk of fracturing. The risk of fracturing may also be determined from a database of past procedures containing paths that prevented a bone from fracturing and paths that failed to prevent the bone from fracturing.

The path characteristics described above may be quantified and used to construct a cost function allowing the paths to be classified according to an objective criterion. A number of mathematical functions may be considered to combine the characteristics in order to effectively represent clinical objectives. The simplest function is a linear combination, in which case the cost function C(T) of a path T takes the form of a weighted sum of the characteristics described above:

$$C ( T ) = {{L}^{K}}_{i = 1} {w}_{i} \cdot {y}_{i} \qquad [ Math . 4 ]$$

where the weights w, make it possible to control the “balance” between the characteristics yᵢ.

Various sets of weights w, with predefined values may form various cost functions that may be used as they are if they prove to fit well with the practitioner's clinical practice. The practitioner is also able to create her or his own cost function by modifying the values of the weights w, in order to give more or less importance to the characteristics yᵢ, in light of the targeted objectives of the procedure in question. This may be done manually, for example through a user interface, or even automatically based on previous procedures. Indeed, for each of the previous procedures, an evaluation (a score) could be assigned to the path used and the characteristics computed. Based on this information, a system of equations (with a number N of equations significantly higher than the number K of characteristics in question) may be created and solved using regression methods. Neural networks could also be used to generate a cost function.

It may be advantageous to use various cost functions representative of various optimization categories (for example “performance” or “safety”). This makes it possible to take into account predefined criteria or criteria customized by the user. A user may then provide an indication, via a user interface, of the cost function to be considered when displaying the candidate paths on the display screen 13.

As illustrated in FIG. 2, the method 100 comprises a step 106 of displaying, on the display screen 13, superposed on the anatomical model, at least some of the remaining candidate paths with, for each displayed candidate path, a means for rapidly viewing a value of the cost function.

It will be noted that the display screen 13 may take various forms: it may for example be a flat LCD computer screen (LCD monitor), but it may also be a screen of an augmented-reality headset, in which case the displayed information may be projected onto the patient directly.

It is conceivable not to display candidate paths for which the cost function is less than a threshold value.

In particular modes of implementation, the means for rapidly viewing the value of the cost function of a displayed candidate path corresponds to a color code: various respective colors are associated with various values of the cost function. Such a display makes it possible to quickly locate the best path according to established clinical criteria and to select it using the user interface. When a path is selected by the user, it is possible to display the quantifications determined for the characteristics of that path with the aim of providing the user with a detailed view of each characteristic used to compute the cost function of the path. Of course, the system offers the ability to choose any candidate path, which allows optimality criteria not captured by the cost function to be managed.

FIG. 5 schematically illustrates the display of a set of candidate paths 31 on the display screen 13. Each candidate path 31 has been represented with a color associated with the value of the cost function computed for the candidate path 31 in question. The color code 30 used is also displayed. In the example shown in FIG. 5, the candidate paths 31 have been represented on the body surface 21 of the patient. When the user selects one of these candidate paths 31, it is possible to display various views of the selected path in the anatomical model (for example in different sectional planes comprising the selected path).

As illustrated in FIG. 2, the method 100 comprises a step 107 of a user selecting, for the target point in question, a path considered to be optimal among the displayed paths.

The selected optimal path may for example be used to configure the movement of a robotic arm suitable for holding or guiding the medical instrument intended to be used to perform the medical procedure.

All the steps of determining 103 a region and a sampling resolution, of removing 104 certain candidate paths, of computing 105 at least one cost function, of displaying 106 candidate paths and of selecting 107 a path may be iterated a plurality of times to refine the selection of the candidate path that will be used to perform the medical procedure. For this purpose, and as illustrated in step 108 of FIG. 2, the user may for example be asked to indicate, via a user interface, whether she or he wishes to refine the selected candidate path.

Thus, in certain modes of implementation, at least two iterations are performed. Advantageously, for a given iteration (different from the first iteration), the new sampling region includes the point of entry corresponding to the path selected in the previous iteration, the new sampling region is defined to be smaller than the sampling region defined for the previous iteration, and the sampling resolution is defined to be finer than the sampling resolution defined for the iteration (this means that the minimum distance separating two candidate points of entry is smaller).

In certain modes of implementation, for a given iteration (different from the first iteration), the new sampling resolution and/or the new sampling region is defined in light of a variability of the cost function computed for the previous iteration in the vicinity of the path selected in the previous iteration. The variability of the cost function may for example be measured as a function of the angles α and β. The higher the variability in the previous iteration, the more recommendable it will be to choose a fine resolution for the new iteration. The lower the variability, the more recommendable it will be to choose an extensive sampling region for the new iteration.

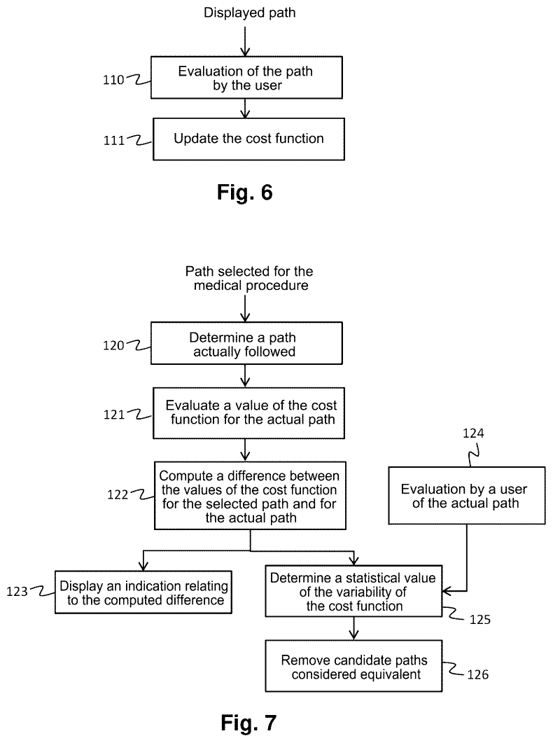

As illustrated in FIG. 6, the method 100 may also comprise a step 110 of the user evaluating at least one displayed candidate path, and a step 111 of updating the cost function based on the obtained evaluation.

For each displayed path, the user may evaluate the path by assigning it a score. This score may be used to trigger an optional update of the parameters of the cost function (for example, if the cost function is a linear combination of a plurality of quantified characteristics, the weights of the linear combination may be modified in order to adapt to the score provided by the user, for example via regression algorithms or neural networks).

The path that was actually followed by the medical instrument during the medical procedure may deviate from the selected candidate path, in particular as a result of imprecision in the placement of the needle or of biomechanical effects (deflection of the needle, movement of the anatomy of interest due to breathing or due to effects associated with insertion). It is therefore relevant to determine the retrospective cost function for the actual path followed by the medical instrument, and a difference ΔC(T) between the value C(T) of the cost function computed for the candidate path selected for the procedure and the value C′(T) of the cost function of the actual path.

Thus, and as illustrated in FIG. 7, the method 100 may also comprise the following steps, after the medical procedure:
determining 120 in the anatomical model an actual path that was actually followed by the medical instrument during the procedure (this actual path may in particular be determined from medical images of the patient acquired at a time when the medical instrument is in place, i.e. when it has reached the target point and is in the right place to perform the treatment);
determining 121 a value of the cost function for the actual path;
computing 122 a difference between the value of the cost function of the candidate path selected for the procedure and the value of the cost function of the actual path.

Computation of the value C′(T) of the cost function of the actual path requires the position of the medical instrument after insertion to be estimated from a control image. This may be done manually (by clicking on the point of entry and on the end of the instrument) or automatically by using image-processing methods to segment the instrument and then extract the required information.

The retrospective determination of the cost function of the actual path may in particular be used as a means for alerting the user of incorrect execution of the procedure (the quality of placement of the instrument is less than planned). This alert may be triggered when the variation ΔC(T) in the cost function is significant. A threshold of significance may be determined from a retrospective analysis of past paths assigned an evaluation criterion (a score).

Thus, and as illustrated in FIG. 7, the method 100 may comprise a step 123 of displaying, on the display screen 13, an indication relating to the value of the difference computed in step 122.

A retrospective analysis of the statistics of the variation ΔC(T) in the cost function for paths followed as planned may be conducted with the aim of determining typical values of ΔC(T), in order to determine path equivalence classes. Specifically, a statistic of ΔC(T) may be derived for a set of paths judged to have been followed as planned in order to determine what is the typical variation in the cost function. This value may then be used in the method 100 for selecting an optimal path, in order to avoid suggesting to a user paths that would be too similar (i.e. paths the cost function of which is not sufficiently different).

Thus, and as illustrated in FIG. 7, the method 100 may comprise the following steps:
obtaining 124 an evaluation, by the user, of the actual path;
determining 125, in light of the computed difference and of the obtained evaluation, a statistical value representative of the variability of the cost function;
removing 126 candidate paths that are considered to be equivalent given the statistical value thus obtained.

In the method 100 for assisting with selection of an optimal path, steps 101 to 108, 110 and 111 described with reference to FIGS. 2 and 6 are all implemented before the medical procedure; steps 120 to 126 described with reference to FIG. 7 are implemented after the medical procedure. These steps therefore do not involve execution of a surgical procedure. Steps 108, 110, 111 and 120 to 126 are optional.

The data-processing device 10 described above makes it possible to suggest to a practitioner a set of optimal or near-optimal paths for a minimally invasive procedure, based on a three-dimensional anatomical model of the patient. The suggested paths take into account constraints of various types (for example practical constraints, performance-related constraints and/or safety constraints). The practitioner is able to choose a path that may be sub-optimal in respect of a predefined criterion, but that is preferable in respect of constraints not addressed by that criterion.

The proposed solution allows fast and optimized exploration of the set of solutions with a view to determining candidate paths (“multi-resolution” approach to sampling candidate points of entry). It allows the user to rapidly view various candidate paths and their optimality criteria. The proposed solution is flexible since it is able to adapt to the specific needs of the procedure (need for increased placement precision, need for increased margins of safety as a result of potential weakness in the patient's condition, etc.). The practitioner is further able to update the optimality criteria (cost function) in light of user feedback (evaluation of a path selected for the procedure).

The above description was given mainly with respect to the case of a single target point (for example for a “single-needle” procedure), or indeed with respect to the case where a plurality of target points are considered in succession in order to determine an optimal path individually for each of the target points (for example for a “multi-needle” procedure). In the case of a procedure in which a plurality of medical instruments must be inserted (for example for a “multi-needle” procedure), it is however also possible to consider the set of target points together (and therefore to consider respective groups of candidate paths associated with various target points). This may in particular make it possible to take into account, in the computation of the cost function, characteristics that depend on candidate paths with respect to one another. For example, this may make it possible to take into account, in the computation of the cost function, a maximum relative angle between the various paths pairwise (it may be advantageous to minimise this relative angle). According to another example, this may make it possible to take into account a difference between a distance separating the various paths pairwise and a target value (the target value may in particular be derived from recommendations of the manufacturer of the medical instrument used).

1. A data-processing device comprising a processor, a computer memory and a display screen, the computer memory comprising program-code instructions that, when they are executed by the processor, configure the processor to implement a method for assisting with selection of at least one optimal path for a minimally invasive medical procedure within an anatomy of interest of a patient,
the processor being configured to:
obtain a three-dimensional anatomical model of the patient from previously acquired medical images of the patient, the anatomical model comprising a representation of the body envelope and of the anatomy of interest of the patient,
identify in the anatomical model at least one target point to be reached in a region to be treated within the anatomy of interest of the patient, and
perform at least one iteration in which the processor is configured to:
determine a sampling region and a sampling resolution for sampling candidate points of entry, in the anatomical model, on the body envelope of the patient, each candidate point of entry belonging to the sampling region, the sampling resolution being representative of a minimum distance separating two candidate points of entry, each pair formed by the target point and a candidate point of entry forming one candidate path,
remove candidate paths in light of at least one predetermined validity criterion,
for each remaining candidate path, quantify each of a plurality of predetermined characteristics, and compute at least one cost function from the quantified characteristics,
display, on the display screen, superposed on the anatomical model, at least some of the remaining candidate paths with, for each displayed candidate path, a means for rapidly viewing a value of the cost function, and
obtain the selection, by a user, for said at least one target point, of a path considered to be optimal among the displayed candidate paths.

2. The device of claim 1, wherein the processor is configured to perform at least two iterations, and wherein, for an iteration of rank n, n being an integer strictly greater than one:
the sampling region of the iteration of rank n is defined to be smaller than the sampling region defined for the iteration of rank (n−1), and to include the candidate point of entry corresponding to the path selected in the iteration of rank (n−1), and
the sampling resolution of the iteration of rank n is defined to be finer than the sampling resolution defined for the iteration of rank (n−1).

3. The device of claim 2, wherein the sampling resolution of the iteration of rank n is defined in light of a variability of the cost function computed for the iteration of rank (n−1) in the vicinity of the path selected in the iteration of rank (n−1).

4. The device of claim 2, wherein the sampling region of the iteration of rank n is defined in light of a variability of the cost function computed for the iteration of rank in the vicinity of the path selected in the iteration of rank (n−1).

5. The device of claim 1, wherein, to sample the candidate points of entry, the processor is configured to determine, in the anatomical model, on the body envelope of the patient, a set of points defined by a spherical coordinate system centered on the target point, each point being defined by a distance r_{i,j} from the target point and by two angles αᵢ and βᵢ the angles αᵢ and βᵢ being defined such that, for indices i and j corresponding to strictly positive integers:
$${α}_{i , j} = {α}_{i - 1 , j} + \frac{R}{{r}_{i , j}}$$
$$and$$
$${β}_{i , j} = {β}_{i , j - 1} + \frac{R}{{r}_{i , j}}$$
where R is representative of a minimum distance separating two candidate points of entry on the body envelope of the patient.

6. The device of claim 1, wherein one validity criterion allows, for a given candidate path, at least one of the following to be verified:
a validity of the length of the candidate path with respect to a medical instrument envisioned for the procedure,
an ability to configure a robotic arm equipped with a medical instrument so that said medical instrument will be able to follow the candidate path,
the presence of objects with which the patient is equipped obstructing the candidate path, and or
an intersection of the candidate path with at least one critical anatomical structure internal to the body of the patient.

7. The device of claim 1, wherein a plurality of validity criteria are considered to remove candidate paths, and the various validity criteria are evaluated in an order defined in light, for each validity criterion, of an estimated computation time required to assess said validity criterion.

8. The device of claim 1, wherein the plurality of characteristics of a candidate path comprises at least one of the following characteristics:
a minimum distance between the candidate path and a critical anatomical structure internal to the body of the patient,
a length of the intersection between the candidate path and the anatomy of interest,
a minimum angle of incidence between the candidate path and an anatomical interface crossed by the candidate path,
an estimated value representative of the stability, under the effect of its own weight, of a medical instrument positioned up to the target point along the candidate path,
a minimum distance between an envelope of the region to be treated and an envelope of an ablation region estimated for the candidate path, and/or
an angular difference between the candidate path and a major axis of the region to be treated.

9. The device of claim 1, wherein a plurality of cost functions are computed from the quantified characteristics, each cost function being computed with a different set of respective weightings assigned to the various quantified characteristics, and the processor is configured to obtain an indication, by a user, of the cost function to be considered when displaying candidate paths.

10. The device of claim 1, wherein the means for rapidly viewing the value of the cost function for each displayed candidate path comprises a color code associating various respective colors with various values of the cost function.

11. The device of claim 1, wherein the processor is configured to display the quantifications determined for the characteristics of the path selected by the user.

12. The device of claim 1, wherein the processor is configured to obtain, for at least one displayed candidate path, an evaluation by the user of said candidate path, and to update the cost function in light of said evaluation.

13. The device of claim 1, wherein the processor is configured to:
determine in the anatomical model, after the procedure, an actual path followed by a medical instrument during the procedure, from medical images of the patient acquired at a time when the medical instrument is in place,
determine a value of the cost function for the actual path, and
compute a difference between the value of the cost function of the candidate path selected for the procedure and the value of the cost function of the actual path.

14. The device of claim 13, wherein the processor is configured to compare the computed difference with a predetermined threshold and to display an indication relating to the value of the computed difference.

15. The device of claim 13, wherein the processor is configured to:
obtain an evaluation of the actual path by the user,
determine, in light of the computed difference and of the obtained evaluation, a statistical value representative of the variability of the cost function, and
remove candidate paths that are considered to be equivalent given the statistical value thus obtained.
